(19) United States
(12) Patent Application Publication
(10) Pub. No.: US 2014/0130197 A1
(43) Pub. Date: May 8, 2014

(54) HYBRID VARIETY H1176 WITH HIGH LYCOPENE

(71) Applicant: H.J. HEINZ COMPANY, (US)

(72) Inventor: Rich OZMINKOWSKI, Lodi, CA (US)

(73) Assignee: H.J. HEINZ COMPANY, Pittsburgh, PA (US)

(21) Appl. No.: 13/853,871

(22) Filed: Mar. 29, 2013

Related U.S. Application Data
(60) Provisional application No. 61/624,147, filed on Apr. 13, 2012.

(51) Int. Cl. A01H 5/08

(52) U.S. Cl. CPC A01H 5/08
USPC 800/260; 800/317.4; 435/411

(57) ABSTRACT

Hybrid tomato variety ‘H1176’ is described. The ‘H1176’ tomato variety is a ground-culture hybrid tomato variety suitable for machine harvest and has high levels of lycopene.

### CROSS-REFERENCE TO RELATED APPLICATIONS

This application claims the benefit of U.S. Provisional Application No. 61/624,147, filed Apr. 13, 2012, which is hereby incorporated by reference in its entirety.

### FIELD

This invention relates to the field of plant breeding. In particular, this invention relates to a new variety of tomato, Solanum lycopersicum, denominated ‘H1176’.

### BACKGROUND

Breeding improved tomato varieties involves providing genetics that give an advantage to the grower, processor, consumer, or other member of the supply chain. The improvement may be in the form of field performance, disease resistance, factory performance, or a fruit quality characteristic. For a tomato variety to be suitable to be grown for processing, the variety must have a concentrated fruit setting and maturity, firm fruit, and sufficient rot tolerance to allow early fruit to remain rot-free while later fruit continues to develop and ripen.

Most commercial processing tomato varieties are hybrids resulting from a cross pollination of two true-breeding, inbred parents. Through the use of true-breeding lines, a hybrid is produced that often displays characteristics of each parent, and often demonstrates characteristics that are superior to either parent alone, or that allow a hybrid to mask inadequacies of the individual parents.

An important contribution that tomatoes provide to the human diet is the antioxidant lycopene. Specifically, processing tomato varieties are the key form of tomato intake in the US diet. Higher levels of lycopene are beneficial both from a nutritional standpoint and from a consumer perception and quality standpoint. Tomato varieties having higher levels of lycopene result in products with a deeper red color that can be considered an indicator of higher product quality. Thus, a tomato variety with higher levels of lycopene can be valuable from both a nutritional standpoint and a quality standpoint. However, to be commercially viable and useful, the tomato variety must perform acceptably as required by any other processing tomato variety.

### SUMMARY

In order to meet this need, the present invention provides a new improved tomato variety ‘H1176’, which contains a particularly high level of lycopene, typically 60% to 80% higher than ordinary tomato varieties. The elevated level of lycopene is demonstrated both in the lycopene content of ‘H1176’, and in the elevated Hunter a/b color value of the cooked tomato juice product of ‘H1176’. The ‘H1176’ tomato variety is a midseason variety with resistance to verticillium race 1, fusarium races 1 and 2, root knot nematode. The fruit is very firm with an average weight of 85 grams per fruit with a good level of rot tolerance that makes it very adaptable for once-over machine harvest in regions such as California.

The characteristics that determine the quality of tomato fruit used for processing are different from that of tomato fruit used for the fresh market. Processing characteristics are commonly tested on samples of tomato juice produced in a way that is well known in the art. For example, tomatoes are cooked in a microwave oven for several minutes, lost water is replaced, and the sample is poured into a pulper-finisher to remove skin and seeds and produce a uniform juice sample. Once cooled to room temperature, various tests can be run on the juice including juice Bostwick (a measure of gross viscosity or consistency), refractometer solids to measure soluble sugars and acids, pH, and color via Hunter a/b score. The Hunter a/b score is an international industry and USDA standard color measurement of tomato products that provides a representation of the color of the product in a single dimensionless unit. The “a” value represents color on the green to red dimension whereas “b” represents the blue to yellow dimension; a higher a/b ratio is associated with more red color and is often considered a superior product.

Tomato varieties contain varying levels of lycopene (Garcia and Barrett, 2006). Lycopene content of tomato juice can be measured using a protocol developed by Anthon and Barrett (2001), which involves an ethanol/hexane extraction followed by quantification using reflectance at 503 nm.

As used herein, tomato variety ‘H1176’, tomato plant ‘H1176’, tomato seed ‘H1176’, and ‘H1176’ all refer to the hybrid tomato variety ‘H1176’, and parts and seeds thereof, having ATTCC Accession Number Y1.

Accordingly, in one embodiment, the present invention is directed to tomato seed designated as ‘H1176’ having ATCC Accession Number Y1. In one embodiment, the present invention is directed to a tomato plant and parts isolated therefrom produced by growing ‘H1176’ tomato seed. In another embodiment, the present invention is directed to a tomato plant and parts isolated therefrom having all the physiological and morphological characteristics of a tomato plant produced by growing ‘H1176’ tomato seed having ATCC Accession Number Y1. In still another embodiment, the present invention is directed to an F₁ hybrid tomato seed, plants grown from the seed, and leaves, ovules, pollen, fruit, cotyledons, embryos, meristems, anthers, roots, root tips, pistils, flowers, stems, calli, stalks, hypocotyla, and pericarps isolated therefrom having ‘H1176’ as a source or parent, wherein ‘H1176’ is grown from ‘H1176’ tomato seed having ATCC Accession Number Y1.

Tomato plant parts include leaves, ovules, pollen, fruit, cotyledons, embryos, meristems, anthers, roots, root tips, pistils, flowers, stems, calli, stalks, hypocotyls, pericarps, and the like. In another embodiment, the present invention is further directed to tomato fruit, stems, leaves, parts of leaves, roots, root tips, pollen, ovules, and flowers isolated from ‘H1176’ tomato plants. In another embodiment, the present invention is further directed to tissue culture or cells derived from ‘H1176’ tomato plants.

In yet another embodiment, the present invention is further directed to a method of selecting tomato plants by a) growing ‘H1176’ tomato plants wherein the ‘H1176’ plants are grown from tomato seed having ATCC Accession Number Y1; and b) selecting a plant from step a). In another embodiment, the present invention is further directed to tomato plants, plant parts and seeds produced by the tomato plants, where the tomato plants are isolated by the selection method of the invention.

In another embodiment, the present invention is further directed to a method of breeding tomato plants by crossing a tomato plant with a plant grown from ‘H1176’ tomato seed having ATCC Accession Number Y1. In still another embodiment, the present invention is further directed to tomato plants, tomato parts from the tomato plants, and seeds produced therefrom where the tomato plant is isolated by the breeding method of the invention.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 illustrates fruit from the tomato variety ‘H1176’.

### DETAILED DESCRIPTION

Described herein is a new and distinct tomato variety named ‘H1176’ that was developed to provide a ground-culture hybrid tomato variety (i.e., not grown on stakes) that is suitable for machine harvest, has high levels of lycopene, is adaptable for machine harvest, and is adaptable to the climactic conditions of regions such as California. Another characteristic of the tomato variety ‘H1176’ is that it is a machine-harvest processing tomato variety that is adapted to regions such as California, USA.

Plants of the tomato variety ‘H1176’ are resistant to verticillium wilt race 1, fusarium wilt races 1 and 2, and root knot nematode. Additionally, plants of the tomato variety ‘H1176’ are dark green in color and medium in size compared to other tomato varieties of the same market class. Fruit from the tomato variety ‘H1176’ is extremely firm. Moreover, the tomato variety ‘H1176’ is adapted to culture in regions such as California. However, ‘H1176’ performs poorly in more humid climates such as the Midwestern region of the USA.

### Characterization of the ‘H1176’ Tomato Variety

### Seedling

Anthocyanin in hypocotyl of 2-15 cm seedling: Absent
Habit of 3-4 week old seedling: Normal

### Mature Plant

### Growth: Determinate

### Form: Normal

Size of canopy (compared to others of similar type): Medium
Habit: Sprawling (e.g., decumbent)

### Stem

Average length (in cm): 76 cm

### Branching: Sparse (e.g., ‘Brehm's Solid Red’, ‘Fireball’)

Branching at cotyledonary or first leafy node: Absent
Number of nodes below first inflorescence: 7-10
Number of nodes between early (1st 2nd, 2nd 3rd) inflorescences: 1-2
Number of nodes between later developing inflorescences: 1-2

### Leaf (Mature Leaf Beneath the 3rd Inflorescence)

### Type: Tomato

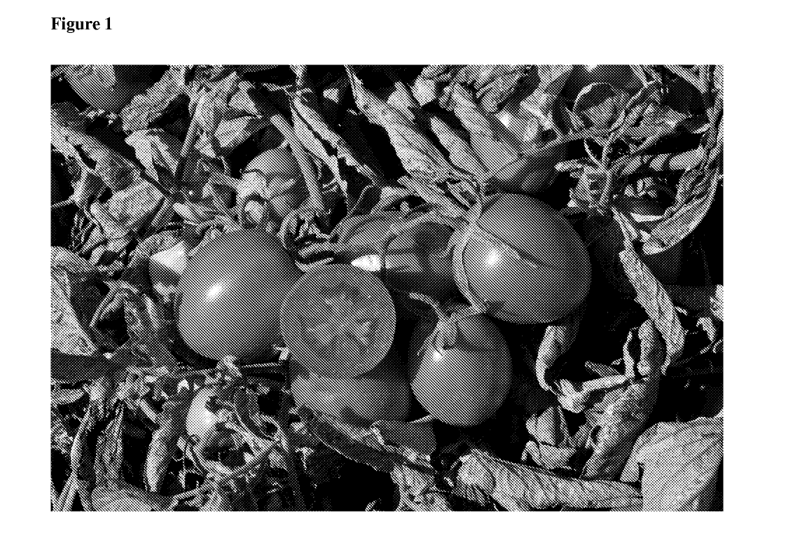

Please describe morphology of mature leaf: #2—compound leaf with both major and minor leaflets (FIG. 1)
Average length and width (in cm): 24×17 cm
Margins of major leaflets: Shallowly toothed or scalloped
Marginal rolling or wiltiness: Strong
Onset of leaflet rolling: Mid-season
Surface of major leaflets: Wrinkled (e.g., creased, bumpy, or veiny)

### Pubescence: Normal

### Inflorescence (Make Observations on 3rd Inflorescence)

### Type: Simple

Average number of flowers in inflorescence: 4
Leafy or “running” inflorescences: Absent

### Flower

Calyx: Normal, lobes awl-shaped
Calyx-lobes: Shorter than corolla
Corolla color: Old gold
Style pubescence: Absent
Anthers: All fused into tube

### Fruit (3rd Fruit of 2nd or 3rd Cluster)

Describe the typical fruit shape: #10—blocky fruit shape (FIG. 1)
Describe the Shape of transverse section: Round
Describe the shape of stem end: Flat
Describe the shape of blossom end: Slightly pointed
Describe the shape of pistil scar: Dot, with the pistil often attached at maturity
Abscission layer: Absent (jointless)
Point of detachment of fruit at harvest: At calyx attachment
Provide the average length (in mm) of dedicel (from joint to calyx attachment): 22 mm
Provide the average length (in mm) of mature fruit (stem axis): 62.5 mm
Provide the average diameter (in mm) of fruit at widest point: 47.5 mm
Provide the average weight (in g) of mature fruit: 85 grams
Number of locules: Two
Fruit surface: Smooth
Fruit base color (mature-green stage): Dark green
Fruit pattern (mature-green stage): Uniform green
Shoulder color if different from base: Dark green
Fruit color, full-ripe: Red
Flesh color full-ripe: Red/Crimson;
Flesh color: Uniform
Locular gel color of table-ripe fruit: Red

### Ripening: Uniform

### Ripening: Outside in

Stem scar size: Medium (e.g., ‘Rutgers’)

### Core: Present

Epidermis color: Yellow

### Epidermis: Normal

Epidermis texture: Tough
Describe the thickness of the pericarp: Thick
Anthocyanin in hypocotyl of 2-15 cm seedling: Present
Habit of 3-4 week old seedling: Normal

### Disease and Pest Reaction

Disease and pest reaction: Resistant to vericillium race 1, fusarium races 1 and 2, root knot nematode, alternaria stem canker, and stemphyllium.

### Chemistry and Composition of Full-Ripe Fruits

TABLE 1

| | Variety | Check Variety 1 | Check Variety 2 |
|---|---|---|---|
| | ‘H1176’ | ‘Heinz 3402’ | ‘Nunhems 6366’ |
| pH | 4.51 | 4.59 | 4.53 |
| Juice bostwick | 10.78 | 12.22 | 15.18 |
| Soluble solids as °Brix | 5.25 | 5.08 | 5.39 |
| Hunter a/b | 2.35 | 2.26 | 2.26 |
| Lycopene (ppm) | 198 | 119 | n/a |

Fruiting season: Medium (e.g., ‘Westover’)
Relative maturity in areas tested: Medium

### Adaptation

### Culture: Field

Principle use(s): Concentrated products
Machine harvest: Adapted
Regions to which adaptation has been demonstrated: California: Sacramento and Upper San Joaquin Valley; and California: Southern San Joaquin Valley & deserts

### Further Embodiments

Additional methods include, but are not limited to, chasing selfs. Chasing selfs involves identifying inbred plants among tomato plants that have been grown from hybrid tomato seed. Once the seed is planted, the inbred plants may be identified and selected due to their decreased vigor relative to the hybrid plants that grow from the hybrid seed. By locating the inbred plants, isolating them from the rest of the plants, and self-pollinating them (i.e., “chasing selfs”), a breeder can obtain an inbred line that is identical to an inbred parent used to produce the hybrid.

Accordingly, another aspect of the present invention relates a method for producing an inbred tomato variety by: planting seed of the tomato variety ‘H1176;’ growing plants from the seed; identifying one or more inbred tomato plants; controlling pollination in a manner which preserves homozygosity of the one or more inbred plants; and harvesting resultant seed from the one or more inbred plants. The step of identifying the one or more inbred tomato plants may further include identifying plants with decreased vigor, i.e., plants that appear less robust than plants of the tomato variety ‘H1176.’ Tomato plants capable of expressing substantially all of the physiological and morphological characteristics of the parental inbred lines of tomato variety ‘H1176’ include tomato plants obtained by chasing selfs from seed of tomato variety ‘H1176.’

One of ordinary skill in the art will recognize that once a breeder has obtained inbred tomato plants by chasing selfs from seed of tomato variety ‘H1176’, the breeder can then produce new inbred plants such as by sib-pollinating, or by crossing one of the identified inbred tomato plant with a plant of the tomato variety ‘H1176.’

### DEPOSIT INFORMATION

A deposit of the tomato variety ‘H1176’ is maintained by HeinzSeed Company, having an address at 2800 South El Dorado, Stockton, Calif. 95206, United States of America. Access to this deposit will be available during the pendency of this application to persons determined by the Commissioner of Patents and Trademarks to be entitled thereto under 37 C.F.R. §1.14 and 35 U.S.C. §122. Upon allowance of any claims in this application, all restrictions on the availability to the public of the variety will be irrevocably removed by affording access to a deposit of at least 2,500 seeds of the same variety with the American Type Culture Collection, (ATCC), P.O. Box 1549, MANASSAS, VA 20108 USA.

Applicants have made available to the public without restriction a deposit of at least 2500 seeds of tomato variety ‘H1176’ with the American Type Culture Collection (ATCC), P.O. Box 1549, MANASSAS, VA 20108 USA, with a deposit on (Date) which has been assigned ATCC number Y1.

The deposit will be maintained in the ATCC depository, which is a public depository, for a period of 30 years, or 5 years after the most recent request, or for the effective life of the patent, whichever is longer, and will be replaced if a deposit becomes nonviable during that period.

1. Tomato seed designated as ‘H1176’ having ATCC Accession Number Y1.

2. A plant produced by growing the seed of claim 1.

3. A plant part from the plant of claim 2.

4. The plant part of claim 3, wherein said part is selected from the group consisting of leaves, ovules, pollen, fruit, cotyledons, embryos, meristems, anthers, roots, root tips, pistils, flowers, stems, calli, stalks, hypocotyls, pericarps, and portions thereof.

5. A tomato plant having all the physiological and morphological characteristics of the tomato plant of claim 2.

6. A plant part from the plant of claim 5.

7. The plant part of claim 6, wherein said part is selected from the group consisting of leaves, ovules, pollen, fruit, cotyledons, embryos, meristems, anthers, roots, root tips, pistils, flowers, stems, calli, stalks, hypocotyls, and pericarps.

8. An F₁ hybrid tomato plant having ‘H1176’ as a parent where ‘H1176’ is grown from the seed of claim 1.

9. A plant part from the plant of claim 8.

10. The plant part of claim 9, wherein said part is selected from the group consisting of leaves, ovules, pollen, fruit, cotyledons, embryos, meristems, anthers, roots, root tips, pistils, flowers, stems, calli, stalks, hypocotyla, pericarps, and portions thereof.

11. Pollen of the plant of claim 2.

12. An ovule of the plant of claim 2.

13. A tissue culture of the plant of claim 2.

14. A method of making tomato seeds comprised of crossing the plant of claim 2 with another tomato plant and harvesting seed therefrom.

15. A method of making tomato variety ‘H1176’, said method comprising selecting seeds from the cross of one ‘H1176’ plant with another ‘H1176’ plant, a sample of ‘H1176’ tomato seed having been deposited under ATCC Accession Number Y1.
